(19) United States
(12) Patent Application Publication
(10) Pub. No.: US 2021/0321919 A1
(43) Pub. Date: Oct. 21, 2021

(54) FLUID COLLECTION DEVICE

(71) Applicant: D-Rev: Design For the Other Ninety Percent, San Francisco, CA (US)

(72) Inventor: George Justus HOFMEYR, Fish Hoek (ZA)

(21) Appl. No.: 17/327,531

(22) Filed: May 21, 2021

Related U.S. Application Data
(63) Continuation of application No. PCT/IB2020/060048, filed on Oct. 27, 2020.

(30) Foreign Application Priority Data
Nov. 1, 2019 (ZA) 2019/07195

(51) Int. Cl. A61B 5/15; A61B 5/157; A61B 5/02

(52) U.S. Cl. CPC A61B 5/150045; A61B 5/02042; A61B 5/157; A61B 5/150251

(57) ABSTRACT

Aspects of the present disclosure provide a fluid collection device including a rigid or semi-rigid structure having a wedge-shaped leading edge to fit under a patient's buttocks while the patent is supine. At least one receptacle is provided in the structure to receive fluid discharged by the patient. A measurement apparatus associated with the at least one receptacle indicates a threshold volume of fluid collected by the fluid collection device.

### CROSS-REFERENCE TO RELATED APPLICATIONS

This application is a continuation of PCT Application No. PCT/IB2020/060048, filed Oct. 27, 2020; which claims priority from South African provisional patent application number 2019/07195 filed Nov. 1, 2019; the contents of which are incorporated herein by reference in their entirety.

### FIELD OF THE INVENTION

The invention relates to a fluid collection device and in particular to a fluid collection device for monitoring the volume of fluid discharged by a patient.

### BACKGROUND TO THE INVENTION

Postpartum bleeding or postpartum haemorrhage (PPH) is typically defined as the loss of more than 500 ml of blood after vaginal birth or 1,000 ml of blood after a caesarean section within the first twenty-four hours following childbirth. It is the most common cause of maternal deaths globally. Therefore, monitoring of blood loss after birth and early diagnosis of PPH is very important for treatment to be instituted timeously.

The most common method of monitoring of blood loss after birth is visual estimation which has been found to be inaccurate, usually under-estimating by a large amount. Other methods include weighing of blood-soaked linen. However, this does not provide ongoing monitoring. More recently a plastic drape such as the BRASS-V Drape™ has been used. This is a disposable plastic drape which is placed beneath the patient after the birth and secured in place with tapes which are tied around the patient's abdomen. Blood and clots, which tend to pool around the patient's buttocks due to indentation of the mattress, are swept manually into a conical transparent measuring receiver which hangs over the end of the bed.

One difficulty with the BRASS-V Drape™ is that the patient has to be raised from the bed or be rolled from side to side to position the drape beneath her. Further, the medical practitioner may encounter some difficulty in removing all the blood from the drape. The final blood loss therefore has to be measured by weighing the drape with blood and subtracting the known dry weight of the drape. This requires an accurate, regularly calibrated scale, and the complexity of the process poses a risk of errors.

The applicant believes there is scope to improve the situation.

The preceding discussion of the background to the invention is intended only to facilitate an understanding of the present invention. It should be appreciated that the discussion is not an acknowledgment or admission that any of the material referred to was part of the common general knowledge in the art as at the priority date of the application.

### SUMMARY OF THE INVENTION

In accordance with the invention there is provided a fluid collection device comprising a rigid or semi-rigid structure having a wedge-shaped leading edge to fit under a patient's buttocks while the patient is supine and at least one receptacle in the structure to receive fluid discharged by the patient, the device including a measurement apparatus associated with the at least one receptacle to indicate a threshold volume of fluid collected by the fluid collection device.

Further features provide for the device to include a first receptacle formed in the structure adjacent the leading edge for receiving the fluid discharged by the patient, and a second receptacle formed in the structure adjacent the first receptacle, wherein the measurement apparatus includes an arrangement by which the first receptacle overflows into the second receptacle, the overflowing being indicative of the threshold volume of fluid collected. The overflowing from the first receptacle to the second receptacle may have the advantage of providing a visual indication of the possibility of postpartum haemorrhage.

Further features provide for the first and second receptacles to be flat-bottomed open basins formed in the rigid or semi-rigid structure; for the rigid or semi-rigid structure to be a tray or pan that includes the first and second receptacles formed in the tray and for the first receptacle to be calibrated by means of gradation marks provided on at least one sidewall thereof to indicate a volume of fluid in the first receptacle. The gradation marks may be steps in the sidewalls of the first receptacle such that an area of a flat bottom of the first receptacle is smaller than that of its open top.

A further feature provides for the second receptacle to be calibrated by means of gradation marks provided on at least one sidewall thereof to indicate a volume of fluid in the second receptacle. The gradation marks may be steps in the sidewalls of the second receptacle such that an area of a flat bottom of the second receptacle is smaller than that of its open top.

Further features provide for the first receptacle to hold about 500 ml of fluid; and for the second receptacle to hold between 500 and 1500 ml of fluid.

Further features provide for the structure to include a groove or narrow gap by which the fluid overflows from the first receptacle to the second receptacle. This may allow fluid to flow to the second receptacle when the first receptacle overflows but reduces fluid transfer between receptacles by accidental swilling of the fluid.

Further features provide for the first and second receptacles to be separated by means of a divider; wherein the divider has a groove or narrow gap therein by which the first receptacle overflows into the second receptacle.

Further features provide for the device to have a consistent height between its bottom and top surfaces, so that the first and second receptacles are horizontally positioned when the structure is placed on a flat horizontal surface.

Further features provide for the device to have a generally rectangular outer profile in plan view with the first and second receptacles forming a major portion of the area within the outer profile; for the first receptacle to be located in one half of the rectangle adjacent the leading edge; for the second receptacle to be located in the other half of the rectangle opposite the leading edge; and for the first receptacle to be oval in plan view, and the second receptacle to be rectangular in plan view.

Further features provide for the second receptacle to be used to temporarily place a placenta following childbirth.

In another aspect of the invention the device includes one receptacle that is a flat-bottomed open basin formed in the rigid or semi-rigid structure, and the measurement apparatus is an upstanding formation centrally located on the flat-bottom of the open basin, the upstanding formation enabling the volume of fluid in the basin to be measured by a height of the fluid relative to the formation, irrespective of an angle of tilt of the structure. The upstanding formation may be a cone having gradation marks spaced along its height. The cone may be integrally formed with the flat bottom of the basin. A top of the cone may represent a measured volume of about 500 ml of fluid thereby indicating a likelihood of postpartum haemorrhage if the cone is submerged. The basin itself, however, may hold at least 1000 ml of fluid.

Further features provide for the outer edges of the structure to include downwardly directed sidewalls that have edges co-planar with flat bottoms of the first and second receptacles, so as to support the device on a flat surface and permit the device to be carried by inserting fingers behind the downwardly directed sidewalls.

In all aspects of the invention the device may be moulded, preferably injection moulded; and may be made from a plastics material. In other embodiments the device may be formed from steel, preferably stainless steel, and may be press-formed. The device may be stackable in a vertical configuration with like devices.

The fluid collected may be blood; the device may be an obstetric fluid collection device for collecting and measuring postpartum blood loss.

Embodiments of the invention will now be described, by way of example only, with reference to the accompanying drawings.

### BRIEF DESCRIPTION OF THE DRAWINGS

In the drawings:

FIG. 1 is a three-dimensional view of a fluid collection device according to one embodiment of the present disclosure;

FIG. 2 is a top view of the fluid collection device of FIG. 1;

FIG. 3 is a front view of the fluid collection device of FIG. 1;

FIG. 4 is a side view of the fluid collection device of FIG. 1;

FIG. 5 is a section view along the line I-I of FIG. 2;

FIG. 6 is a detailed view of the portion A in FIG. 5;

FIG. 7 is a side view of three fluid collection devices stacked in a vertical configuration;

FIG. 8 is a section view along the line II-II of FIG. 7;

FIG. 9 is a section view along the line of FIG. 7;

FIG. 10 is a three-dimensional view of the fluid collection device of FIG. 1 positioned under a patient's buttocks while the patient is supine;

FIG. 11 is a three-dimensional view of a fluid collection device according to another embodiment of the present disclosure;

FIG. 12 is a top view of the fluid collection device of FIG. 11;

FIG. 13 is a side view of the fluid collection device of FIG. 11;

FIG. 14 is a three-dimensional view of a fluid collection device according to yet another embodiment of the present disclosure;

FIG. 15 is a side sectional elevation of the device of FIG. 14 showing fluid therein; and

FIG. 16 is similar to FIG. 15 but with the device tilted.

### DETAILED DESCRIPTION WITH REFERENCE TO THE DRAWINGS

Aspects of the present disclosure provide a fluid collection device including a rigid or semi-rigid structure having a leading edge shaped to fit under a patient's buttocks while the patent is supine. The device includes a measurement apparatus associated with at least one receptacle to indicate a threshold volume of fluid collected by the fluid collection device.

In some embodiments the structure includes two receptacles formed therein. A first receptacle is formed adjacent the leading edge for receiving fluid discharged by the patient and a second receptacle is formed adjacent the first receptacle. The first and second receptacles are arranged so that the first receptacle overflows into the second receptacle. The overflowing may be indicative of a condition such as postpartum haemorrhage.

In other embodiments the structure includes one receptacle that is a flat-bottomed open basin formed in the rigid or semi-rigid structure. In such embodiments, the measurement apparatus may be an upstanding formation centrally located on the flat bottom of the open basin. The upstanding formation may enable the volume of fluid in the basin to be measured by a height of the fluid relative to the formation, irrespective of an angle of tilt of the structure. A threshold volume of fluid collected in the receptacle may indicate a likelihood of postpartum haemorrhage.

Exemplary embodiments of a fluid collection device are now described in greater detail with reference to the accompanying figures.

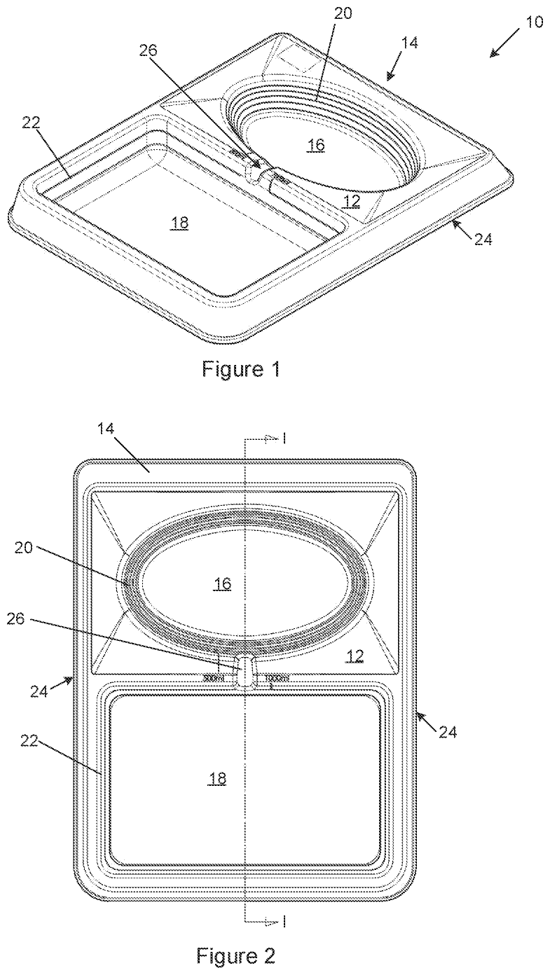

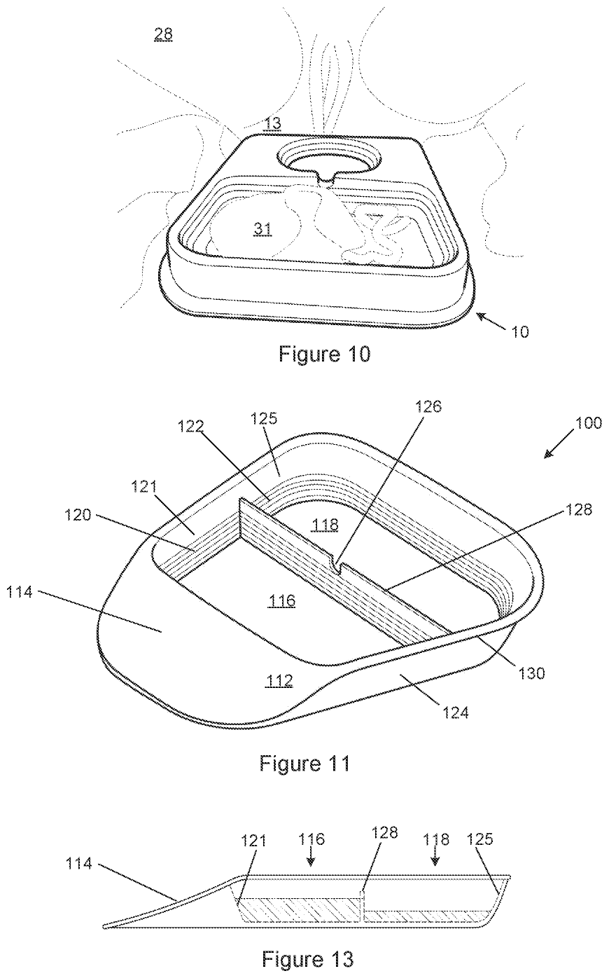

FIGS. 1 to 10 illustrate one exemplary embodiment of a fluid collection device (10). The fluid collection device (10) includes a generally rectangular rigid or semi-rigid structure (12) having a wedge-shaped leading edge (14) along a width thereof, shaped to fit under a patient's buttocks (13) while the patient is supine, as illustrated in FIG. 10. The leading edge (14) may be disposed at an angle of between 10° to 60° to a horizontal plane through a top surface of the structure (12). In this embodiment, the leading edge (14) is disposed at an angle of about 60° as shown in greater detail in FIG. 6. All that is required, however, is that the leading edge be capable of being tucked under a patient's buttocks while the patient is supine, so many other shapes (generally wedge-shapes), configurations and/or angles of leading edges could be used.

The structure (12) includes two receptacles (16, 18) which, in this embodiment, are flat-bottomed open basins formed in the structure. The receptacles (16, 18) form a major portion of an area of the structure (12) as most clearly shown in FIG. 2. A first receptacle (16) is located in one half of the structure (12) adjacent the leading edge (14), for receiving fluid discharged by the patient, and a second receptacle (18) is located in the other half of the rectangular structure (12), opposite the leading edge (14).

The first receptacle (16) may have different shapes, such as the illustrated oval shape in plan view, and may be calibrated by means of gradation marks (20) provided on a sidewall (21) thereof to indicate a volume of fluid therein. In this embodiment, the first receptacle (16) holds about 500 ml of fluid. That threshold is the amount below which bleeding from a vaginal birth does not generally indicate post-partum haemorrhage.

While any form of gradation marks (20) may be used, in the illustrated embodiment the gradation marks (20) are steps formed in the sidewalls (21) of the first receptacle (16) such that an area of a flat bottom (23) of the first receptacle (16) is smaller than its open top as more clearly shown in FIG. 6. The second receptacle (18) may also have different shapes and in this embodiment is rectangular in plan view. It is also calibrated by means of gradation marks (22) provided on a sidewall (25) thereof to indicate a volume of fluid therein and may hold between 500 and 1500 ml of fluid. Like the first receptacle (16), the gradation marks (22) of the second receptacle (18) may be steps in the sidewalls of the second receptacle (18) such that an area of a flat bottom (27) of the second receptacle (18) is smaller than its open top as more clearly shown in FIG. 5.

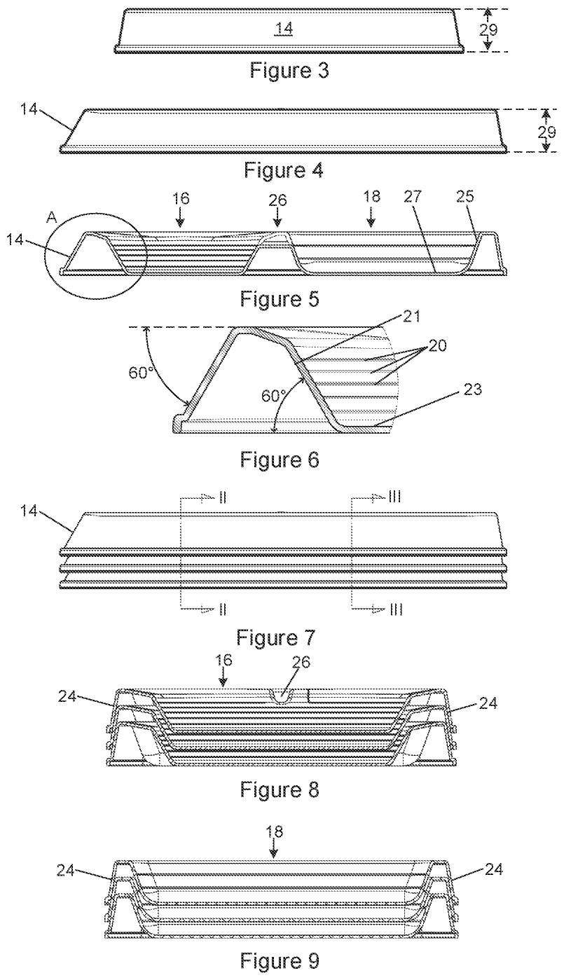

Outer edges of the structure (12) may include downwardly directed sidewalls (24) that have edges co-planar with flat bottoms (23, 27) of the first and second receptacles (16, 18), so as to support the device (10) on a flat surface and permit the device (10) to be carried by inserting fingers behind the downwardly directed sidewalls (24). The structure (12) may have a consistent height (29) between its bottom and top surfaces, so that the first and second receptacles (16, 18) are horizontally positioned when the structure is placed on a flat horizontal surface as more clearly shown in FIGS. 3 and 4.

The structure (12) includes a groove or narrow gap (26) by which fluid overflows from the first receptacle (16) to the second receptacle (18). The groove (26) is located at a gradation mark (20) which reads 500 ml, which indicates that the first receptacle (16) holds its maximum volume of 500 ml before it overflows into the second receptacle (18). Overflowing may be indicative of a possibility of postpartum haemorrhage. The narrow gap (26) also functions to reduce fluid transfer between receptacles by accidental swilling of the fluid.

In use, during childbirth, a medical practitioner has to be alert to the possibility of postpartum haemorrhage where bleeding continues beyond the normal bleeding associated with childbirth (about 500 ml). The wedge-shaped leading edge (14) of the fluid collection device (10) is tucked beneath a patient's (28) buttocks (13) without the patient (28) having to roll over or lift her buttocks (13). Fluid discharged by the patient (28) is collected in the first receptacle (16). In this embodiment the fluid is blood. The first receptacle (16) has gradations allowing the medical practitioner to easily monitor the amount of blood that has been collected by reading the volume on the gradation marks (20). The first receptacle (16) is configured to hold 500 ml of blood and when it overflows into the second receptacle (18) it is obvious to the medical practitioner that over 500 ml of blood has been lost by the patient (28). Hence a visual indication of the possibility of postpartum haemorrhage is provided. As illustrated in FIG. 10, the second receptacle also provides a convenient place to temporarily place the placenta (31) following childbirth.

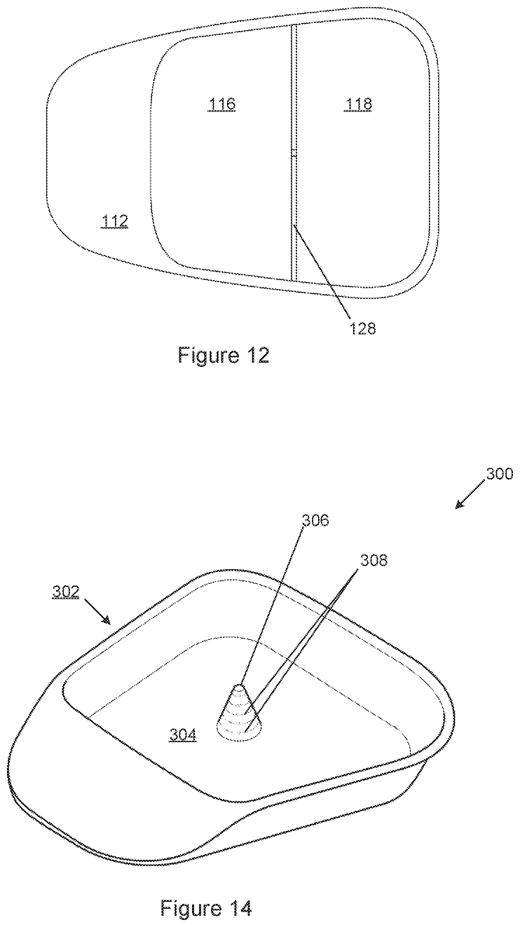

A second embodiment of a fluid collection device (100) is illustrated in FIGS. 11 to 13. The fluid collection device (100) functions in a substantially similar manner to the fluid collection device (10) of the first embodiment. The fluid collection device (100) includes a generally rectangular rigid or semi-rigid structure (112) having a wedge-shaped leading edge (114) along its width thereof, shaped to fit under a patient's buttocks while the patient is supine. The structure (112) includes two receptacles (116, 118) or in this embodiment, flat-bottomed open basins formed therein with the receptacles (116, 118) forming a major portion of the area of the structure (112) as most clearly shown in FIG. 12. A first receptacle (116) is located in one half of the structure (112) adjacent the leading edge (114), for receiving fluid discharged by the patient, and a second receptacle (118) is located in the other half of the structure (112), opposite the leading edge (114).

The first receptacle (116) is calibrated by means of gradation marks (120) provided on a sidewall (121) thereof to indicate a volume of fluid therein and holds about 500 ml of fluid, as shown in FIGS. 11 and 13. The second receptacle (118) is calibrated by means of gradation marks (122) provided on a sidewall (125) thereof to indicate a volume of fluid therein and hold between 500 and 1000 ml of fluid as shown in FIG. 13. The structure (112) includes a groove or narrow gap (126) by which fluid overflows from the first receptacle (116) to the second receptacle (118). The groove (126) is located at a gradation mark (120) which reads 500 ml which indicates that the first receptacle (116) holds its maximum volume of 500 ml before it overflows into the second receptacle (118). The overflowing may be indicative of a possibility of postpartum haemorrhage.

This fluid collection device (100) has a differently shaped leading edge (114) than the first embodiment, and includes first and second receptacles (116, 118) that are separated by means of a divider (128) which has a groove or narrow gap (126) by which the first receptacle (116) overflows into the second receptacle (118). In this second embodiment, the first and second receptacles (116, 118) have a rounded rectangular shape in plan view and the structure (112) includes a lip (130) which is roughly co-planar with flat a top of the first and second receptacles (116, 118), the lip (130) permitting the device (100) to be easily carried by user.

FIG. 13 shows a side view of the device (100) with the slope of the wedge-shaped leading edge (114) being disposed at an angle of about 20° to a horizontal plane through the top surface of the structure (112), for a substantially more acutely angled wedge shape than the embodiment of FIGS. 1 to 10. The location of the first and second receptacles (116, 118) with the divider (128) and groove (126) are indicated with dotted lines. In FIG. 13, the shading indicates how the first receptacle (116) can fill up, before the fluid flows through the groove (126) into the second receptacle (118). In this illustration the first receptacle (116) is full, and the second receptacle (118) is approximately half full, which may indicate that the patient has lost approximately 750 ml of fluid.

The second embodiment of the fluid collection device (100) may be used in substantially the same manner as the first embodiment of the fluid collection device (10) described above.

In some embodiments the fluid collection device may be moulded, preferably injection moulded and may be made from a plastics material. The plastic material renders the fluid collection device relatively light, however, the device is still be sufficiently robust to sustain the weight of a patient and to give a stable support during childbirth.

In other embodiments, the fluid collection device may be made from a rigid material such as steel, preferably stainless steel, in which case it could be made by press-forming. In these embodiments the fluid collection device may be re-usable.

Further, it is envisaged that edges and interior corners of the device may be rounded to prevent harm to the patient and/or medical practitioner, or tearing of surgical gloves during use. The device may be stackable in a vertical configuration with like devices as illustrated in FIGS. 7 to 9. The fluid may be blood, and the device may be an obstetric fluid collection device for collecting and measuring postpartum blood loss, and wherein the overflow of the first receptacle to the second receptacle indicates a possibility of postpartum haemorrhage.

FIGS. 14 to 16 illustrate a third embodiment of a fluid collection device (300). In this embodiment, the device (300) includes only one receptacle (302) that is an open basin (303) with a flat bottom (304), formed in the rigid or semi-rigid structure of the device (300). The measurement apparatus is an upstanding formation (306), which in this embodiment is shaped as a cone with its apex projecting upwards the flat bottom (304). The cone (306) is centrally located on the flat bottom (304) of the open basin (303). The cone (306) enables the volume of fluid in the basin to be measured by a height of the fluid therein relative to the cone (306). To facilitate accurate measurement, the cone (306) may be provided with gradation marks (308) spaced along its height. In this embodiment, the fluid is blood and the cone has five gradation marks (308)—100 ml, 200 ml, 300 ml, 400 ml and 500 ml. The top of the cone measures the 500 ml volume. This means that if the cone (306) is submerged, it is obvious to a medical practitioner that over 500 ml of blood has been lost by the patient. Hence a visual indication of a possibility of postpartum haemorrhage is provided. The basin itself may hold at least twice this amount of fluid, for example 1000 ml.

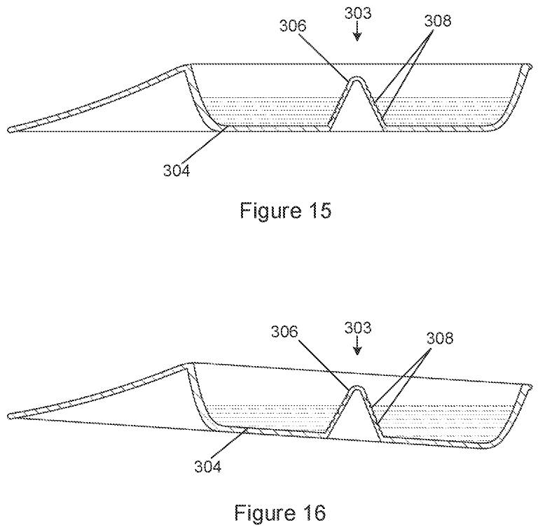

Locating the upstanding formation centrally within the open basin (303) has the advantage that the volume of fluid measured as a height up the cone is unaffected by a tilt of the basin. This is illustrated in FIGS. 15 and 16, where the device (300) is shown horizontally and tilted at an angle. Because the cone (306) is located at the approximate centre point of the basin (303), straight lines representing the top of the fluid at different tilt angles all intersect at this point, so the measured height is the same irrespective of the tilt in any direction.

The fluid collection device of the present disclosure may have the advantage in that it is inexpensive, easy to clean and reusable. It is also convenient to put in place below a patient's buttocks without substantially moving the patient. Further, the fluid collection device of the present disclosure is efficient in collecting most of the blood loss from the patient following childbirth, resulting in less blood accumulating around the patient's hips and back in use compared with using a soft drape. It enables a medical practitioner to easily monitor volume of fluid (e.g. blood, urine and/or amniotic fluid) loss, on an ongoing basis. In routine clinical use, the gradated receptacles or gradations in the cone would be sufficiently accurate to give an objective measure of blood loss. Alternatively, the fluid in the receptacles could be poured into a measuring jug to give a more accurate reading.

It is appreciated that numerous variations may be made to the embodiments of the invention described above without departing from the scope hereof. For example, in other embodiments the fluid collection device may be provided with more than two receptacles, the volume of each receptacle being used to quickly determine clinically important amounts of blood or other fluid loss. Each divider between such receptacles may have a groove or slot to allow fluid to flow between the receptacles when the desired volume is reached, but may reduce fluid transfer between receptacles by accidental swilling of the fluid. Instead of a cone-shaped upstanding formation, upstanding formations such as a narrow cylindrical shape could be used.

The foregoing description has been presented for the purpose of illustration; it is not intended to be exhaustive or to limit the invention to the precise forms disclosed. Persons skilled in the relevant art can appreciate that many modifications and variations are possible in light of the above disclosure.

The language used in the specification has been principally selected for readability and instructional purposes, and it may not have been selected to delineate or circumscribe the inventive subject matter. It is therefore intended that the scope of the invention be limited not by this detailed description, but rather by any claims that issue on an application based hereon. Accordingly, the disclosure of the embodiments of the invention is intended to be illustrative, but not limiting, of the scope of the invention, which is set forth in the following claims.

Finally, throughout the specification and accompanying claims, unless the context requires otherwise, the word ‘ comprise’ or variations such as ‘ comprises’ or ‘ comprising’ will be understood to imply the inclusion of a stated integer or group of integers but not the exclusion of any other integer or group of integers.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A fluid collection device comprising a rigid or semi-rigid structure having at least one receptacle in the structure to receive fluid discharged by the patient, the device including a measurement apparatus associated with the at least one receptacle to indicate a threshold volume of fluid collected by the fluid collection device, wherein the device further includes a first receptacle formed in the structure adjacent the leading edge for receiving the fluid discharged by the patient, and a second receptacle formed in the structure adjacent the first receptacle, wherein the measurement apparatus includes an arrangement by which the first receptacle overflows into the second receptacle, the overflowing being indicative of the threshold volume of fluid collected.

2. The fluid collection device as claimed in claim 1, having a wedge-shaped leading edge to fit under a patient's buttocks while the patient is supine.

3. The fluid collection device as claimed in claim 1, wherein the device has a generally rectangular outer profile in plan view with the first and second receptacles forming a major portion of the area with the outer profile, with the first receptacle located in one half of the rectangle adjacent the leading edge, and the second receptacle located in the other half of the rectangle opposite the leading edge.

4. The fluid collection device as claimed in claim 1, wherein the structure includes a groove or narrow gap by which the fluid overflows from the first receptacle to the second receptacle.

5. The fluid collection device as claimed in claim 1, wherein the first and second receptacles are flat-bottomed open basins formed in the rigid or semi-rigid structure.

6. The fluid collection device as claimed in claim 1, wherein the rigid or semi-rigid structure is a tray that includes the first and second receptacles formed in the tray.

7. The fluid collection device as claimed in claim 1, wherein the first receptacle is calibrated by means of gradation marks provided on at least one sidewall thereof to indicate a volume of fluid in the first receptacle.

8. The fluid collection device as claimed in claim 1, wherein the second receptacle is calibrated by means of gradation marks provided on at least one sidewall thereof to indicate a volume of fluid in the second receptacle.

9. The fluid collection device as claimed in claim 1, wherein the first receptacle holds about 500 ml of fluid.

10. The fluid collection device as claimed in claim 1, wherein the first receptacle holds between 500 and 1500 ml of fluid.

11. The fluid collection device as claimed in claim 1, wherein the first and second receptacles are separated by means of a divider, wherein the divider has a groove or narrow gap therein by which the first receptacle overflows into the second receptacle.

12. The fluid collection device as claimed in claim 1, wherein the first receptacle is oval in plan view, and the second receptacle is in rectangular plan view.

13. The fluid collection device as claimed in claim 1, wherein the device has a consistent height between its bottom and top surfaces, so that the first and second receptacles are horizontally positioned when the structure is placed on a flat horizontal surface.

14. The fluid collection device as claimed in claim 1, wherein the device includes one receptacle that is a flat-bottomed open basin formed in the rigid or semi-rigid structure, and the measurement apparatus is an upstanding formation centrally located on the flat bottom of the open basin, the upstanding formation enabling the volume of fluid in the basin to be measured by a height of the fluid relative to the formation, irrespective of an angle of tilt of the structure.

15. The fluid collection device as claimed in claim 14, wherein the upstanding formation is a cone having gradation marks paced along its height.

16. The fluid collection device as claimed in claim 15, wherein the come is integrally formed with the flat bottom of the open basin.

17. The fluid collection device as claimed in claim 15, in which the top of the cone represents a measured volume of about 500 ml of fluid, while the basin holds at least 1000 mL of fluid.

18. The fluid collection device as claimed in claim 1, wherein outer edges of the structure include downwardly directed sidewalls that have edges co-planar with flat bottoms of the first and second receptacles, so as to support the device on a flat surface and permit the device to be carried by inserting fingers behind the downwardly directed sidewalls.

19. The fluid collection device as claimed in claim 1, wherein the device is moulded and made from a plastics material or is made from metal and is press-formed.

20. The fluid collection device as claimed in claim 1, wherein the fluid is blood, the device is an obstetric fluid collection device for collecting and measuring postpartum blood loss, and wherein the threshold volume of fluid collected indicates a possibility of postpartum haemorrhage.
